(19) United States
(12) Patent Application Publication
(10) Pub. No.: US 2022/0313828 A1
(43) Pub. Date: Oct. 6, 2022

(54) Use of low-molecular glycosidically bound terminal galactosides and fucosides for bonding to toxins that act as galectins in the treatment of intoxications, in particular ricin intoxications

(71) Applicant: ILMA BIOCHEM GMBH, Munster (DE)

(72) Inventor: Katharina HOLTKAMP, Cloppenburg (DE)

(21) Appl. No.: 17/607,946

(22) PCT Filed: Dec. 13, 2019
(86) PCT No.: PCT/DE2019/000326

§ 371 Date: Nov. 1, 2021

(30) Foreign Application Priority Data
May 15, 2019 (DE) 10 2019 003 442.2

(51) Int. Cl. A61K 47/54

(52) U.S. Cl. CPC A61K 47/549; A61K 47/545

(57) ABSTRACT

The invention relates to the use of low-molecular glycosidic compounds with terminal D-galactose and L-fucose in the treatment of intoxications with ricin. The identified compounds form a lectin bond with the B chain of the ricin and thereby prevent further endocytosis of the toxins. The compounds should be ingested as early as possible after incorporation of the toxin and can also be used preventively. The groups bonded to the D-galactose and L-fucose can be other saccharides (e.g. fructose), polyalcohols (e.g. sorbitol), diacyl-glycerides or flavonoids (e.g. quercetin).

Galactose-heteropolysaccharide hydrolysates such as guar gum flour can also be used. The identified compounds can be used for oral, pulmonary and systemic intoxications. They should be used in corresponding pharmaceutical forms of administration. The compounds are dosed in molar excess.

Ricin is an extremely toxic protein from the seeds of the castor oil plant (Ricinus communis). Chemically, ricin consists of a cell-bonding B chain and a toxin-mediated A chain. The B chain is a so-called lectin, i.e., a structure that attaches to glycopeptides. Ricin lectin belongs to the group of so-called galectins, as it attaches specifically to the galactose peptides of cell membranes and thus makes it possible for the AB toxin complex to be absorbed into the interior of the cell. Galectins occurring physiologically in the body have important tasks relating to the signal transduction, growth regulation and the absorption of substances into the cell.

The actual poisonous effect of the ricin is caused by the A chain: After the two chains are split in the Golgi apparatus, the Golgi apparatus acts as N-glycoside hydrolase on the eukaryotic ribosome. Here, an adenine molecule is split off on the so-called sarcin-ricin loop, which leads to the loss of function of the ribosome. A single ricin A chain can catalytically destroy up to 1500 ribosomes per minute. 0.25 milligrams of isolated ricin is sufficient for a fatal intoxication of an adult.

Other known toxins with galactose-specific lectin chains that bond to cell surfaces to initiate endocytosis include abrin from the rosary pea (Abrus precatorius) (UniProtKB-P11140), modeccin from the Adenia digitata (UniProtKB-Q6RUL6), the Shiga toxin subunit B UniProtKB-Q7BQ98), the Shiga-like toxin 1 subunit B (UniProtKB-P69179) and the diphtheria toxin (UniProtKB-Q6KE85).

Promising options for treating ricin intoxications only exist before the ribosomes are inactivated. This inactivation begins with a significant delay compared to the point in time at which the toxin was absorbed, so that there is a sufficient window of therapy. However, patients do not display the first symptoms until the ribosomes have been inactivated, which means that patients initially appear symptom-free during this therapeutic window, making the symptoms more difficult to recognize. A possible therapy would also have to take into account whether the toxin was absorbed orally, through inhalation or parenterally.

There are only a few specific therapy options for ricin intoxications. A vaccine consisting of a modified antibody against the A chain was developed to protect against ricin intoxication (U.S. Pat. No. 9,133,253B2). The vaccine can only be used prophylactically for people at risk. To treat an acute intoxication, a monoclonal antibody against the A chain was developed as well (U.S. Pat. No. 5,626,844A), but it is only effective in the therapeutic window between the ingestion of the toxin and the endocytosis. The disaccharide lactose found in milk and milk products can reduce a ricin intoxication as well (1). Epigallocatechin gallate likewise inhibits the toxic effect on human macrophage cells in vitro (2).

So far, only a few research papers on the affinity inhibition of the bonding of the ricin B chain to the cell wall have been published (3). Some of the methods used are considered out of date (4). There are, however, extensive scientific studies on the inhibition of galectins that occur physiologically in humans: The effect of a 1,4 β-D-galactomannan hydrolysate on colon cancer, patented by Galectin Therapeutics, is described (5). The substance that was studied is not yet available as an approved drug, however. Furthermore, the galectin inhibitor N-(1-deoxy-D-lactulos-1-yl)-L-leucine was successfully tested on breast cancer metastases (6) as well as modified citrus pectin (GCS-100) as a galectin-3 inhibitor (7,8). The research status was summarized in 2013 by Téllez-Sanz et al. A total of 36 substances are presented, mostly galactose and lactose derivatives with aromatics (9).

Galectin-3 inhibitors can also be used for non-alcoholic steatohepatitis (NASH), for inflammatory reactions and for fibroses. Different galactosides (patents US20080089959A1 and SE0100172D0) and hydrolysates from galactose heteroglycans (U.S. Pat. No. 9,974,802B2) have been developed for this purpose. The disaccharide lactulose (4-O-β-D-galactopyranosyl-D-fructofuranose) can also be used to prevent the accretion of Rotaviruses on cell surfaces (patent CA2520647A1).

Since it is not yet known which of the previously known 14 physiological galectins is mimicked by the ricin B chain, this research cannot yet be conclusively assessed with regard to its significance for the possible treatment of ricin intoxications. It could be galectin-4, however, due to the two galactose detection regions of the ricin B chain. The compounds, identified so far predominantly as galectin-3 inhibitors, were included in the research. Other glucosides, galactosides and fucosides were included in the research as well.

A commercially available lateral flow assay was used to quantify the inhibition of the lectin bonding of the B chain on the cell surface. The bonding partner mimicking the cell surface is the specific glycopeptide asialofetuin. Dawson et al. (10) evaluated this model in a microtiter plate variant as a suitable in vitro test system for this purpose.

In addition, cytotoxicity tests were carried out with Vero cell lines. The cells were comparable to (11) sensitive to ricin and were incubated at different ricin concentrations with the compounds to be studied in different concentrations. It was possible to determine the specific inhibition coefficients of the individual compounds in this regard.

The results of the in vitro studies showed the highest inhibition coefficients for low-molecular weight compounds with terminal pyranose-D-galactose bound β-glycosidically in position 1 (CAS No. 59-23-4) or terminal pyranose-L-fucose bound α-glycosidically in position 1 (CAS No. 2438-80-4). Other sugars, glucosamines, sialic acids and glucuronic acids showed no or an insufficient inhibitory effect. The variants not bound in position 1 and the non-terminal galactose and fucose compounds did not show an adequate inhibitory effect either.

The residues glycosidically bound to D-galactose or L-fucose in the manner described in para. [0011] can be:
a. Monosaccharides, especially D-fructose (lactulose) and D-mannose bound to the respective carbon-4
b. Disaccharides and oligosaccharides, especially lactose bound to carbon 2′ (2′ fucosyllactose) or 3 (3 fucosyllactose)
c. Sugar alcohols, especially D(−)sorbitol (lactitol)
d. Diacylglycerides, in particular 1,2-diacyl-sn-glycerol bound to position 3
e. Flavonols, especially quercetin, bound to carbon 3 (quercetin galactoside)
f. Anthocyanins, especially cyanidin, bound to carbon 3 (cyanidin galactoside)
g. Flavanols, especially (−)epicatechin, bound to carbon 3 (epicatechin galactoside).

Furthermore, hydrolysates of polysaccharides obtained from galactose heteroglycans (arabinogalactans, guaran, carubin and karaya), obtained by acid hydrolysis, have an adequate inhibitory effect.

The inhibitory effect of the compounds with a sufficient inhibitory effect follows a logarithmic correlation between the inhibitory effect and the amount of the inhibiting compound used, i.e., large excesses of the inhibiting compound (up to two molar solutions) must be used. Since the compounds referenced in para. [0012] are food ingredients (guaran, for example), approved food supplements (quercetin galactoside, for example) or food additives (2′-fucosyllactose, lactitol, for example) or also already established drugs (lactulose), they can be used in higher concentrations.

The solution principle according to the invention involves the use of the compounds referenced in para. [0012] in the event of a ricin intoxication or an intoxication with the other toxins mentioned in para. [0003] to prevent a potential incorporation of the toxins as soon as possible after their incorporation has been detected. A sufficient inhibition of galectin can be achieved in the first 30 minutes after the incorporation. An application even up to 12 hours after the incorporation may still be useful.

The solution principle according to the invention includes the use of oral pharmaceutical preparations (aqueous solutions, chewable tablets) of the compounds referenced in para. [0012] together with the excipients required in the event of an oral intoxication.

The solution principle according to the invention includes the use of pulmonary pharmaceutical preparations (pulmonary sprays) of the compounds referenced in para. [0012] together with the excipients required in the event of a pulmonary intoxication. Disaccharide lactulose, which is already approved as an excipient (humectant) in asthma sprays, is particularly suitable in this regard.

The solution principle according to the invention relates to the use of the compounds referenced in para. [0012], which are absorbed enterally after an oral administration but not or only partially metabolized in orally administrable pharmaceutical preparations and in parenterally administrable pharmaceutical preparations for intoxications in which the toxins have already reached the vascular system.

Embodiment of an oral pharmaceutical preparation according to para. [0015]:

100 g guar gum flour is heated with 900 ml of 5% phosphoric acid for 30 minutes at 80° C. and then neutralized with a sodium hydroxide solution (pH 7.2).

Dosage: Take 200 ml after ingestion of the toxin. Take 50 ml 3 times a day for oral intoxication prophylaxis.

Embodiment of a pharmaceutical preparation to be inhaled according to para. [0016]:

5% solution of lactulose in purified water, sterile filtered, filled into a pulmonary application form (pressure spray container).

Dosage: Apply 10 to 20 sprays after a pulmonary ingestion of the toxin. Apply 2 sprays every hour for prophylaxis.

Embodiment of an oral pharmaceutical preparation according to para. [0017]:

Tablets with 500 mg quercetin galactoside/tablet

Dosage: Take up to 10 tablets. Take 2 tablets 3 times a day for prophylaxis.

Embodiment of a parenteral pharmaceutical preparation according to para. [0017]:

A 5% lactulose solution in purified water is sterilized and filled as an infusion.

Dosage: Infuse 500 ml of the infusion solution within 30 minutes. Infuse 100 ml several times a day for prophylaxis.

### Patent Citations

| Publication | Priority | Publication | | |
|---|---|---|---|---|
| number | date | date | Assignee | Title |
| U.S. Pat. No. 9,133,253B2 | 2010 Apr. 22 | 2013 Nov. 7 | US Secretary | Ricin vaccine |
| | | | of Army | and methods of |
| | | | | making thereof |
| U.S. Pat. No. 5,626,844A | 1991 Nov. 4 | 1997 May 6 | US Secretary | Monoclonal |
| | | | of Army | antibody against |
| | | | | ric A chain |
| US20080089959A1 | 2003 Apr. 7 | 2008 Apr. 17 | La Jolla | Composition and |
| | | | Pharmaceutical | uses of galecti |
| | | | Co | antagonists |
| | | | GlycoGenesys | |
| | | | Inc | |
| SE0100172D0 | 2001 Jan. 22 | 2001 Jan. 22 | Worldwide | New inhibitors |
| | | | applications | against galectins |
| CA2520647A1 | 2003 Mar. 28 | 2004 Nov. 4 | Bristol Myers | Compositions |
| | | | Squibb Co | containing |
| | | | | lactulose for |
| | | | | treating rotavirus |
| | | | | infections |
| U.S. Pat. No. 9,974,802B2 | 2011 Dec. 28 | 2017 Jul. 20 | Galectin | Composition of |
| | | | Therapeutics Inc | novel carbohydrate |
| | | | | drug for treatment |
| | | | | of human diseases |

### Non-Patent Literature Cited

(1) R. Rasooly, X. He, M. Friedman, Milk inhibits the biological activity of ricin, J. Biol. Chem. 287 (33) (2011) 27924-27929, http://dx.doi.org/10.1074/jbc.M112.362988 (http://www.ncbi.nlm.nih.gov/pubmed/22733821).
(2) P. D. R. Dyer, et al., An in vitro evaluation of epigallocatechin gallate (eGCG) as a biocompatible inhibitor of ricin toxin,
(3) Biochim. Biophys. Acta (2016), http://dx.doi.org/10.1016/j.bbagen.2016.03.024
(4) Lambert J M, McIntyre G, Gauthier M N, Zullo D, Rao V, Steeves R M, Goldmacher V S, Blattler W A., The galactose-bonding sites of the cytotoxic lectin ricin can be chemically blocked in high yield with reactive ligands prepared by chemical modification of glycopeptides containing triantennary N-linked oligosaccharides. Biochemistry.

1991 Apr. 2; 30 (13): 3234-47.
(5) GL Nicolson, J. Blaustein, The interaction of Ricinus communis agglutinin with normal and tumor cell surfaces, Biochim. Biophys. Acta 226 (1972) 543-547 (http://www.ncbi.nlm.nih.gov/pubmed/4338881).
(6) Klyosov A A, Platt D, Zomer E. (2003) Preclinical Studies of

Anticancer Efficacy of 5-Fluorouracil when Co-Administered with the 1,4-β-D-Galactomannan. PRECLINICA, 1,175-186
(7) Glinsky G V, Price J E, Glinsky V V, Mossine V V, Kiriakova G, Metcalf J B (1996), Inhibition of human breast cancer metastasis in nude mice by synthetic glycoamines. Cancer Res. 1; 56 (23): 5319-24
(8) Streetly M J, Maharaj L, Joel S, Schey S A, Gribben J G, Cotter F E (2010) GCS-100, a novel galectin-3 antagonist, modulates MCL-1, NOXA, and cell cycle to induce myeloma cell death, Blood vol. 115 no. 19 3939-3948
(9) Téllez-Sanz R, Garcia-Fuentes L, Vargas-Berenguel A, (2013) Human Galectin-3 Selective and High Affinity Inhibitors. Present State and Future Perspectives. Current Medicinal Chemistry 20: 2979-2990
(10) Dawson, R M, Alderton M R, Wells D, Hartley P G, (2006) Monovalent and polyvalent carbohydrate inhibitors of ricin bonding to a model of the cell-surface receptor. J. Appl. Toxicol. 26: 247-252
(11) Pauly D, Worbs S, Kirchner S, Shatohina O, Dorner M B, et al. (2012) Real-Time Cytotoxicity Assay for Rapid and Sensitive Detection of Ricin from Complex Matrices. PLoS ONE 7 (4): e35360

What is claimed is:

1. Use of chemical compounds with
a. terminal pyranose-D-galactose (CAS No. 59-23-4) β-glycosidically bound at position 1 or
b. terminal pyranose-L-fucose (CAS No. 2438-80-4) α-glycosidically bound at position 1
for bonding to galactoside detection regions of toxins that act as galectin in the treatment of intoxications.

2. Use according to claim 1, characterized in that the residues glycosidically bound to D-galactose or L-fucose in the manner described are selected from the group of saccharides, sugar alcohols and diacyl-glycerides that are formed from
a. monosaccharides, in particular D-fructose and D-mannose bound to the respective carbon 4
b. disaccharides and oligosaccharides, especially lactose bound to carbon 2′ or 3
c. sugar alcohols, especially D(−)sorbitol
d. diacylglycerides, in particular 1,2-diacyl-sn-glycerol bound to position 3.

3. Use according to claim 1, characterized in that the residues glycosidically bound to D-galactose or L-fucose in the manner described are selected from the group of flavonoids, which are formed from
a. flavonols, especially quercetin, bound to carbon 3
b. anthocyanins, especially cyanidin, bound to carbon 3
c. flavanols, especially (−)epicatechin, bound to carbon 3.

4. Use according to claim 1, characterized in that hydrolysates of polysaccharides obtained by acid hydrolysis are selected from the group of galactose heteroglycans which is formed from arabinogalactans, guar, carubin and karaya.

5. Use according to claim 1 for inhibiting bonding to galactoside detection regions of a group of toxins which act as galectin and which, in particular, is formed from the toxins ricin (UniProt: P02879; CAS No. 9009-86-3), abrin (UniProtKB-P11140), modeccin (UniProtKB-Q6RUL6), the Shiga toxin subunit B (UniProtKB-Q7BQ98), the Shiga-like toxin 1 subunit B (UniProtKB-P69179) and diphtheria-toxin (UniProtKB-Q6KE85).

6. Use according to claim 1 to prevent the bonding of galactose-specific lectin of the toxins to terminal N-acetyl galactosamine residues or 1,4-bound galactose units of glycoproteins and glycolipids of the cell surface and the endocytosis triggered thereby in the cell. This endocytosis is a prerequisite for the further toxic effect of the toxins as inhibitors of the ribosomal peptide biosynthesis.

7. Use according to claim 1 for the inhibition of the specific detection regions of the toxins up to 12 hours, preferably up to 30 minutes after an ingestion of the toxins to prevent endocytosis and prophylactic use by people at risk.

8. Use of compounds according to claim 1 in orally administrable pharmaceutical preparations for bonding the toxins in the gastrointestinal tract after an oral ingestion of the toxins.

9. Use of compounds according to claim 1 in pharmaceutical preparations for pulmonary administration for bonding the toxins after a pulmonary ingestion of the toxins.

10. Use of compounds according to claim 1 which are absorbed enterally after oral administration but not or only partially metabolized in orally administrable pharmaceutical preparations and of compounds according to claim 1 in parenterally administrable pharmaceutical preparations for bonding the toxins in the vascular system.
